(19) United States
(12) Patent Application Publication
(10) Pub. No.: US 2021/0308480 A1
(43) Pub. Date: Oct. 7, 2021

(54) LIGHTING ASSEMBLY

(71) Applicant: OSRAM OLED GmbH, Regensburg (DE)

(72) Inventor: Stefan LORENZ, Schwarzenbruck (DE)

(21) Appl. No.: 17/268,873

(22) PCT Filed: Sep. 11, 2019
(86) PCT No.: PCT/EP2019/074199

§ 371 Date: Feb. 16, 2021

(30) Foreign Application Priority Data
Sep. 12, 2018 (DE) 10 2018 122 283.1

(51) Int. Cl. A61N 5/06; H05B 45/20

(52) U.S. Cl. CPC A61N 5/0618; A61N 2005/0651; A61N 2005/0663; H05B 45/20

(57) ABSTRACT

The invention relates to a lighting assembly which comprises a lighting device for emitting blue light, and a controller for operating the lighting device, the controller being designed to adjust the melanopic effect factor of the blue light.

A lighting assembly is specified.

One task to be solved is to specify a lighting assembly that is particularly suitable for so-called “human centric lighting”, in which the human being is the focus of the lighting design.

According to at least one embodiment of the lighting assembly, the lighting assembly comprises a lighting device. The lighting device is intended and configured to emit light during operation. In particular, the lighting device is configured to emit blue light. Blue light is light that is perceived as blue light by human observers, for example under daylight conditions. In particular, blue light may be light that is within the CIE 1931 color spaces in a chromaticity coordinate range with CX≤0.32 and CY≤0.33.

In particular, the blue light is designed to mimic the color of the blue sky. For this purpose, the blue light can in particular be light that lies in a chromaticity coordinate range with CX<0.27 and CY<0.27 in the CIE 1931 color spaces.

To emit the blue light, the lighting device may comprise multiple light sources that are operable independently of each other. For example, the light sources of the lighting device may be or comprise light emitting diodes. For example, the light emitting diodes may each be or comprise one or more light emitting diode chips.

According to at least one embodiment of the lighting assembly, the lighting assembly comprises a controller for operating the lighting device. The controller is a device with which different operating states of the lighting device can be set. For example, the controller can be used to select an operating state from a predetermined group of operating states. For this purpose, the controller may comprise at least one microcontroller and/or at least one integrated circuit. The controller may be arranged separately from the lighting device. In this context, it is possible that one controller is configured to control a plurality of lighting devices of the lighting assembly. Further, it is possible that the controller is integrated into the lighting device, for example into a housing of the lighting device. The controller may further be configured to supply the lighting device with the operating current necessary to operate the lighting device.

According to at least one embodiment of the lighting assembly, the controller is configured to adjust the melanopic effect factor of the blue light emitted by the lighting device. The melanopic effect factor a_{mel,V} is a measure of the influence of a light source on the circadian rhythm and is composed of the quotient of the spectral distribution of the emitted light evaluated with a biological effect spectrum Sₘₑₗ and the same spectral distribution evaluated with the human spectral sensitivity curve V(λ). The melanopic effect factor is defined, for example, in the following standards: DIN SPEC 5031-100:2015, CIE TN 003:2015. With the controller, it is now possible, for example, to control light sources of the illumination device in such a way that when blue light is emitted, this light comprises a desired, adjustable melanopic effect factor.

According to at least one embodiment of the lighting assembly, the lighting assembly comprises a lighting device for emitting blue light and a controller for operating the lighting device. Thereby, the controller is configured to adjust the melanopic effect factor of the blue light.

The lighting assembly described here is based on the following considerations, inter alia. “Human Centric Lighting” attempts to create biologically, visually and emotionally effective light. Often these different aspects require different types of light in the same application. In particular, the stimulation of the photoreceptors in the eye, which have an influence on the circadian rhythm of humans, as a biological effect requires light that is not necessarily visually or emotionally ideal. In this context, the photoreceptors in the eye include the protein melanopsin as a receptor pigment. Through this protein, melatonin secretion can be influenced. Melatonin is a hormone in the circadian, i.e. diurnal rhythm, control circuit of humans, which influences waking and sleeping times. A change of the stimulus on the photoreceptors (also ipRGC stimulus, wherein ipRGC means intrinsic photosensitive regional ganglion cells), conventionally also always causes a change of the light, i.e. for example of the light color.

A high activation of the photoreceptors in the eye is achieved, for example, with light with a high spectral content in the range of blue or cyan, which is visually perceived as cold-white. A low activation is associated with a reduced blue component, which is achieved by illumination with warm-white light. A change in activation, i.e. stimulation of the photoreceptors in the eye, is thus accompanied in this case by a change in light color and color temperature.

With the lighting assembly described here, it is now possible to generate blue light of different melanopic effect factors.

According to at least one embodiment of the lighting assembly, the controller is configured to control the lighting device in such a way that in different operating states of the lighting device, blue light with different melanopic effect factors is emitted by the lighting device with the same chromaticity coordinate of the blue light. That is, without changing the color impression of the generated blue light, it is possible to generate light with different melanopic effect factors. In this context, blue light with “the same” chromaticity coordinate means that a change in the chromaticity coordinate is hardly or not perceived by the human observer. For example, the chromaticity coordinate changes at most in the range of 3 MacAdam ellipses.

In this way, for example, a lighting assembly is realized that creates the illusion of a blue or bluish sky and at the same time makes different levels of activation, i.e. different melanopic effect factors, adjustable without visibly changing the illusion of the blue or bluish sky. In this regard, the ipRGC activation potential, i.e., the melanopic effect factor, of the emitted blue light can be changed by changing the spectrum of the emitted light without changing the chromaticity coordinate of the emitted light.

The blue light emitted by the lighting assembly is not primarily used for illumination, but an emotional level is served by the blue light that is not addressable in conventional white-emitting lighting assembly.

The lighting assembly is configured to produce at least two spectra of blue light that are metameric, that is, for which there is no visually perceptible color difference. However, the at least two different spectra are optimized for increased melanopic efficacy on the one hand or reduced melanopic efficacy on the other hand. Surprisingly, it has also been shown that by using blue light, the biological efficacy can be varied to a much greater extent than would be possible with white light.

According to at least one embodiment of the lighting assembly, the controller is configured to control the lighting device in such a way that, in different operating states of the lighting device, blue light with different chromaticity coordinates is emitted by the lighting device with the same melanopic effect factor of the blue light. In this way, it is possible, for example, for the user of the lighting assembly to adjust the blue hue of the emitted light to a preferred chromaticity coordinate without changing the melanopic effect of the emitted light. Thus, the emitted light can be adapted to the individually preferred coloration.

According to at least one embodiment of the lighting assembly, the lighting device comprises a first light source configured to emit blue light. For example, the first light source may comprise a light emitting diode that emits unconverted blue light. That is, the blue light is generated in particular directly by the operation of one or more light emitting diode chips, without conversion by a conversion element comprising a phosphor. As a result, the generated light comprises a particularly low spectral width. For example, the first light source emits blue light at a peak wavelength between at least 445 nm and at most 480 nm.

According to at least one embodiment of the lighting assembly, the lighting device comprises a second light source configured to emit green-blue light. In this context, the second light source comprises, for example, a light emitting diode that is free of a conversion element. That is, the green-blue light is generated in particular directly by the operation of one or more light-emitting diode chips, without conversion by a conversion element comprising a phosphor. As a result, the generated light comprises a particularly low spectral width. A peak wavelength of the green-blue light is greater than the peak wavelength of the blue light of the first light source and lies, for example, in a range between at least 485 nm and at most 495 nm.

According to at least one embodiment, the lighting device comprises at least one third light source configured to emit light substantially in the spectral range from green to red light. “Substantially” in this context means that the maximum intensity of electromagnetic radiation with a wavelength smaller than 500 nm in the spectrum of the light of the third light source is at most one third of the maximum intensity in the spectrum of the third light source. The light from the third light source can be used to influence a saturation of the blue light generated by the lighting assembly in operation. For example, by adding the third light source, the saturation can be reduced so that the light color is changed from deep blue to the grayish-blue range.

The light from the third light source is characterized by a particularly wide spectral width. For example, the half-width can be at least 170 nm. In particular, the half-width of the spectrum of the light of the third light source is greater than the half-width of the spectrum of the light of the first light source and greater than the half-width of the spectrum of the light of the second light source.

To this end, the third light source may comprise a conversion element configured to substantially fully convert blue light to lower energy light. That is, the third light source comprises, for example, one or more light emitting diode chips that emit blue light during operation. This blue light is converted into light of other colors by a downstream conversion element. The conversion element can comprise different types of phosphors so that the blue light is converted into green, yellow and/or red light, for example. In this case, the conversion element is selected to be thick enough, for example, so that no or hardly any blue light leaves the third light source, but is completely converted into lower-energy light.

The lighting device can comprise several third light sources, which can differ from each other, for example, with regard to the phosphors used in the conversion element.

In particular, it is possible for the lighting device to comprise two different first light sources, a second light source and two different third light sources. The two third light sources may differ from each other, in particular with regard to the proportion of red light in their emission spectrum. For example, the two first light sources differ with respect to the peak wavelength of the generated blue light. Thus, a first light source may generate blue light at a peak wavelength that is smaller by at least 10 nm, in particular at least 20 nm, than the peak wavelength of the blue light of the other first light source. The light sources of the lighting device are operable independently of each other.

According to at least one embodiment of the lighting assembly, the lighting device is provided to emit the blue light from a first emission side and white light from a second emission side facing away from the first emission side. In this way, the lighting assembly makes it possible, on the one hand, to emit light with which a specific melanopic effect is to be achieved. On the other hand, white light can be emitted, in particular in a different direction than the blue light.

According to at least one embodiment of the lighting assembly, the lighting assembly is intended to emit the blue light in the direction of a ceiling of a room. That is, the blue light is not intended for direct illumination, but for indirect illumination. For example, the blue light can be used to create the illusion of a blue sky on the ceiling of a room. The blue light is adjustable in terms of its melanopic effect. Blue scattered light, for example, reflects on the ceiling, hits the user of the lighting assembly and unfolds its melanopic effect there.

According to at least one embodiment of the lighting assembly, the lighting device is arranged to emit the white light in a direction away from a ceiling of a room. On the side away from the ceiling, the lighting device in this embodiment of the lighting assembly may emit the light into the room to be used for illumination therein. The melanopic effect of the total light emitted by the lighting device can be adjusted by changing the melanopic effect factor of the blue light, without changing the light color of the blue light or the white hue of the white light.

In the following, the lighting assembly described herein will be explained in more detail with reference to exemplary embodiments shown in the accompanying figures.

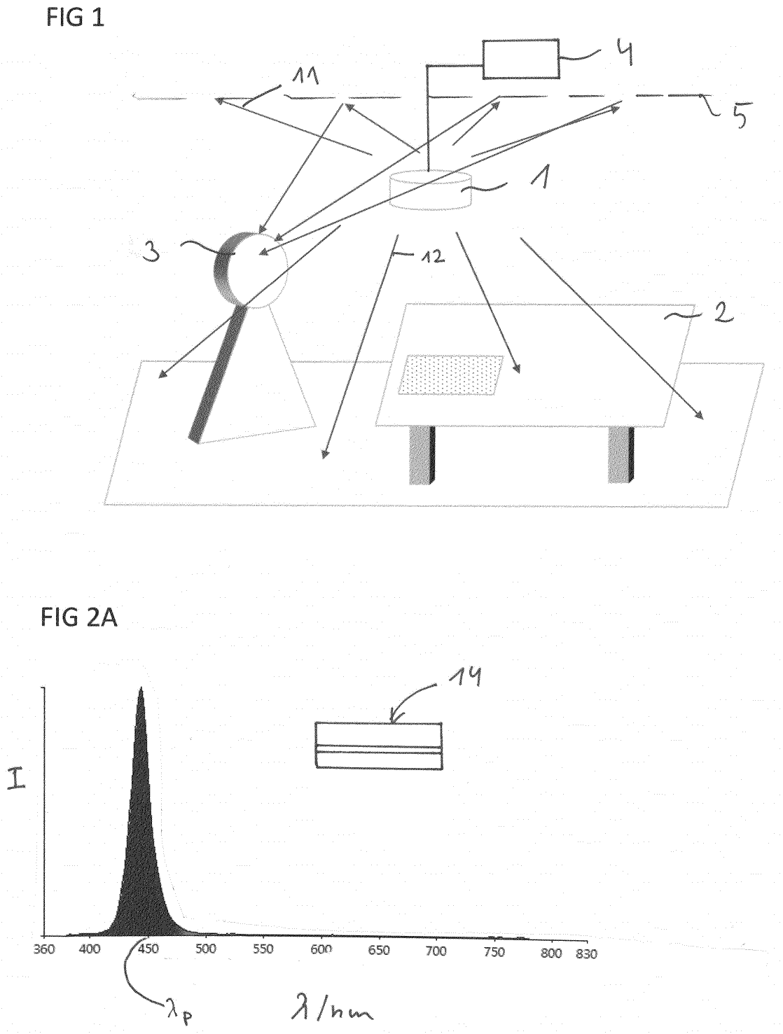

In FIGS. 1, 2A, 2B, 2C, 3A, 3B, 3C, 3D, 4A, 4B, 4C, 5A, 5B, 5C, a lighting assembly described herein is explained by means of schematic diagrams and graphical applications.

Elements that are identical, similar or have the same effect are given the same reference signs in the figures. The figures and the proportions of the elements shown in the figures are not to be regarded as to scale. Rather, individual elements may be shown exaggeratedly large for better representability and/or for better comprehensibility.

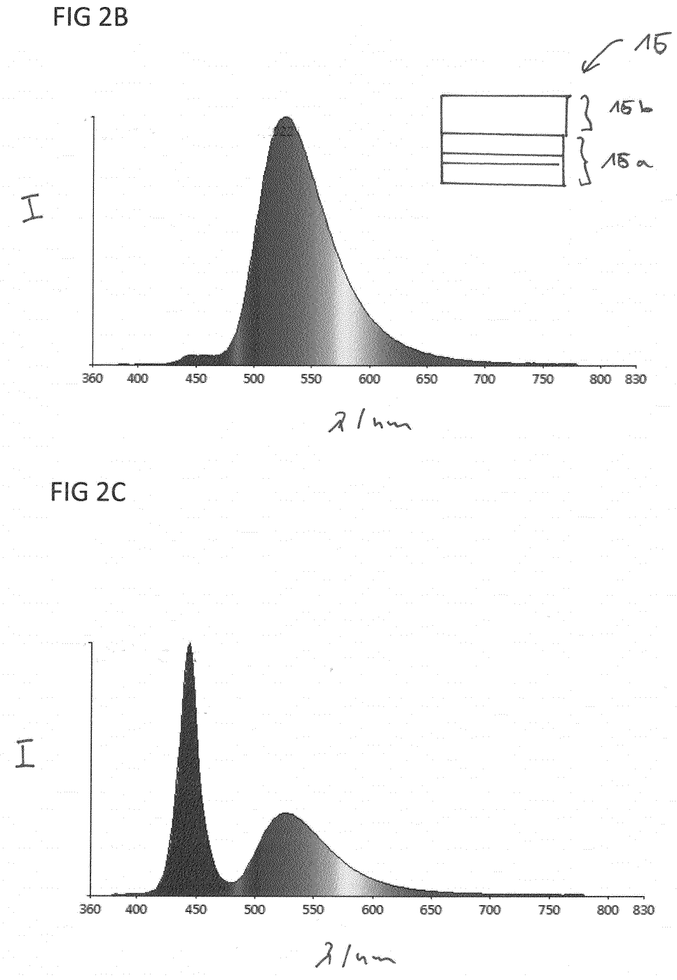

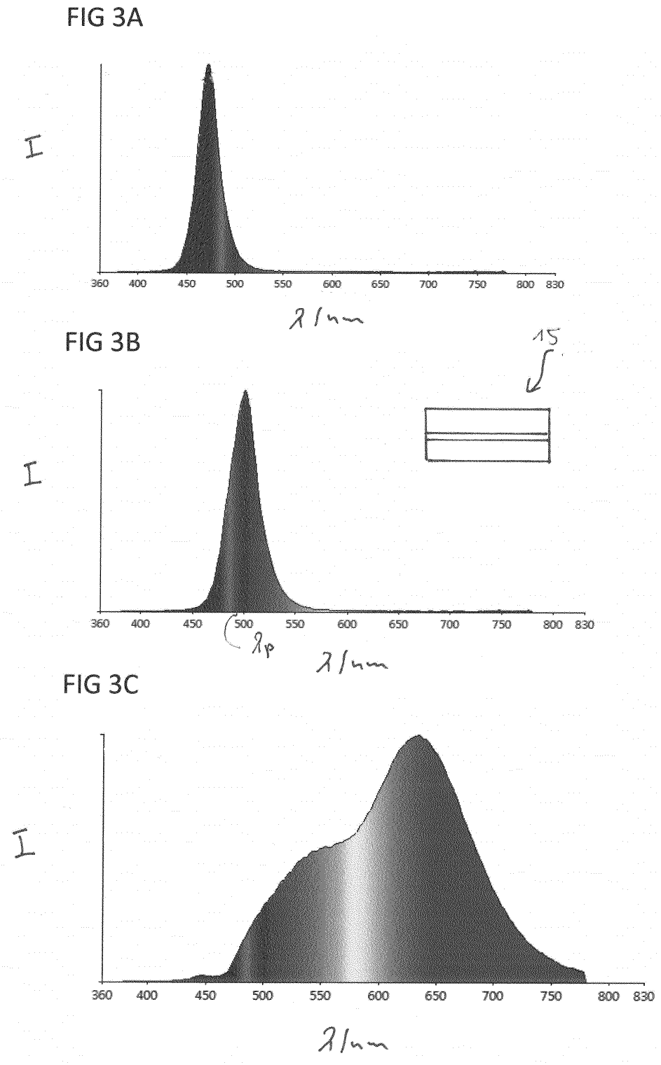

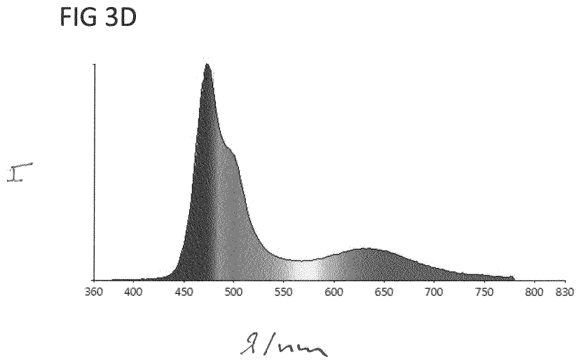

FIG. 1 shows a first exemplary embodiment of a lighting assembly described herein. The lighting assembly comprises a lighting device 1. The lighting device 1 comprises a plurality of light sources 14, 15, 16 (see for example FIGS. 2A, 2B and 3B). In operation, the lighting device 1 emits blue light 11, for example towards the ceiling 5 of a room. Furthermore, it is possible that the lighting device emits white light 12 on its side facing away from the ceiling 5 into the room, for example towards an object 2 and a person 3. The blue light 11 emitted by the lighting device 1 in operation may be reflected or scattered by the ceiling 5 and in this way emitted into the room, for example towards the person 3.

The lighting arrangement further comprises a controller 4, for operating the lighting device 1. The controller 4 can, for example, operate the light sources 14, 15, 16 of the lighting device independently of one another. The controller is thereby configured to adjust the melanopic effect factor of the blue light 11.

This can be done, for example, as explained in association with the graphical representations of FIGS. 2 to 5.

The lighting device may comprise, as schematically shown in FIG. 2A, a first light source 14 which is, for example, a light emitting diode comprising at least one light emitting diode chip. In operation, the first light source 14 generates the blue light shown by means of a spectrum with a peak wavelength λp of less than 450 nm, in particular about 446 nm. In FIGS. 2 to 5, the optical radiant power I per wavelength is plotted in arbitrary units against the wavelength λ.

The lighting device further comprises a third light source 16, FIG. 2B, comprising, for example, a semiconductor body 16a and a conversion element 16b. In operation, blue light can be generated in the semiconductor body 16a, which is substantially completely converted by the conversion element 16b into light having a wavelength of ≥500 nm.

Combining the light from the first light source 14 and the third light source 16 produces the spectrum shown in FIG. 2C with a chromaticity coordinate CIEXY=(0.218/0.262) and a melanopic efficiency factor a_{mel,V}=1.10.

In this lighting device, as described in association with FIGS. 3A to 3D, a second light source 15, compare FIG. 3B, can now be connected, which comprises a peak wavelength λp at approximately 500 nm, in particular 490 nm. In operation, the first light source 14 of FIG. 3A generates the blue light shown by means of a spectrum with a peak wavelength λp of approximately 475 nm.

Furthermore, the lighting device comprises a further third light source 16, compare FIG. 3C. In contrast to the third light source 16 of FIG. 2B, the further third light source is selected with an increased proportion in the spectral range of red light. In this way, light can be produced at the same chromaticity coordinate as for the spectrum of FIG. 2C, wherein the melanopic efficiency factor is 2.11, almost twice as high as for the spectrum of FIG. 2C. This is realized with the spectrum of FIG. 3D. This spectrum is obtained by superimposing the spectra of FIGS. 3A, 3B, 3C.

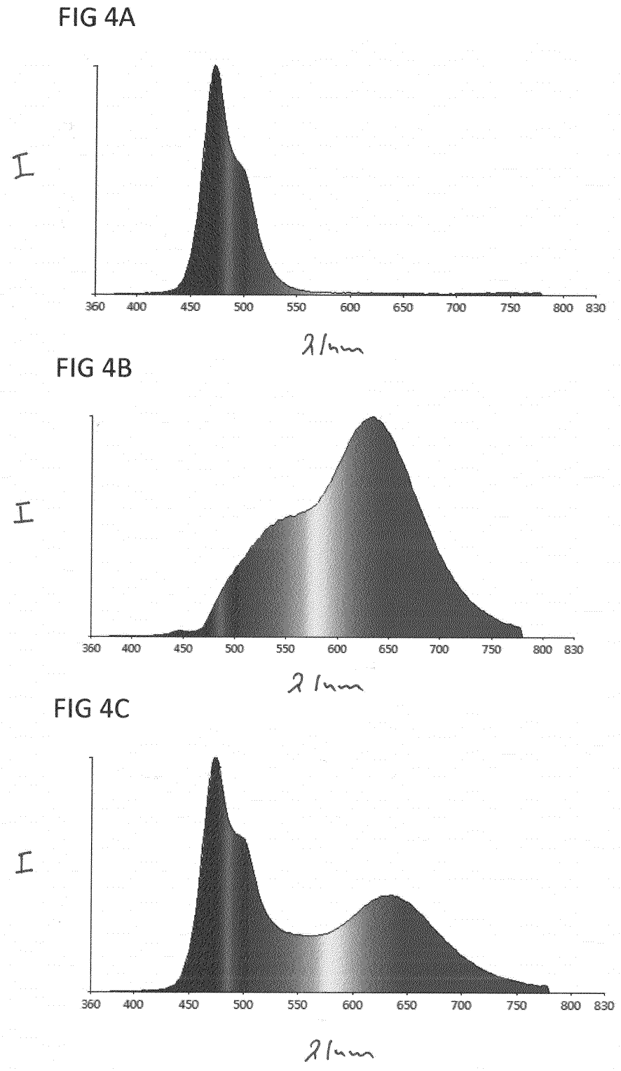

For the light of the spectrum of FIG. 4C, the light of the first light source 14 of FIG. 3A and the second light source 15 of FIG. 3B are combined to the spectrum of FIG. 4A. This spectrum is combined with the spectrum of the further third light source 16 of FIG. 3C to form the spectrum of FIG. 4C. The result is blue light 11 with a chromaticity coordinate CIEXY=(0.313/0.329), a correlated color temperature of approximately 6500 Kelvin and a color rendering index CRI of 57 with a melanopic efficacy factor of 1.26.

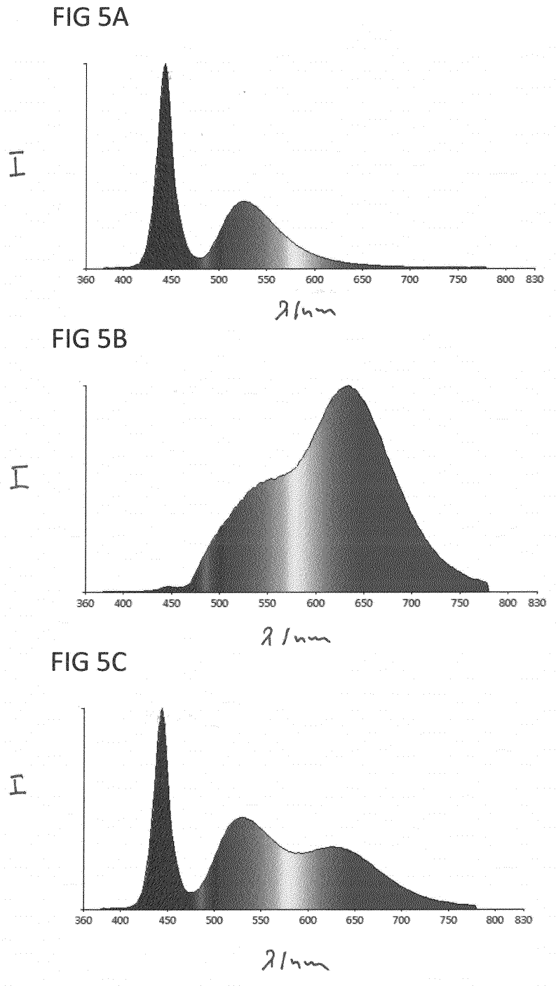

For the light of the spectrum of FIG. 5C, the spectrum of FIG. 2C resulting from the light of the first light source 14 of FIG. 2A and the third light source 16 of FIG. 2B is combined with the spectrum of the further third light source of FIG. 3C. The result is blue light 11 at the same chromaticity coordinate and color temperature as for the spectrum of FIG. 4C. The color rendering index CRI is 81 and the melanopic efficiency factor is 0.736.

The features and embodiments described in association with the figures may be combined in accordance with further exemplary embodiments, although not all combinations are explicitly described. Furthermore, the exemplary embodiments described in association with the figures may alternatively or additionally comprise further features according to the description in the general part.

This patent application claims priority to German patent application 102018122283.1, the disclosure content of which is hereby incorporated by reference.

The invention is not limited to the exemplary embodiments by the description thereof. Rather, the invention encompasses any new feature as well as any combination of features, which in particular includes any combination of features in the patent claims, even if that feature or combination itself is not explicitly specified in the patent claims or exemplary embodiments.

### LIST OF REFERENCE SIGNS

1 lighting assembly
11 blue light
12 white light
14 first light source
16 second light source
16 second light source
16a semiconductor body
16b conversion element
2 object
3 person
4 controller
5 Room ceiling

1. Lighting assembly comprising
a lighting device for emitting blue light, and
a controller for operating the lighting device, wherein
the controller is configured to adjust the melanopic effect factor of the blue light, and
the lighting device is intended to emit the blue light from a first emission side and white light from a second emission side facing away from the first emission side.

2. Lighting assembly according to claim 1, wherein the controller is configured to control the lighting device in such a way that, in different operating states of the lighting device, blue light with different melanopic effect factors is emitted by the lighting device with the same chromaticity coordinate of the blue light.

3. Lighting assembly according to claim 1,
wherein the controller is configured to control the lighting device in such a way that, in different operating states of the lighting device, blue light with different chromaticity coordinates is emitted by the lighting device with the same melanopic effect factor of the blue light.

4. Lighting assembly according to claim 1,
wherein the lighting device comprises at least a first light source configured to emit blue light.

5. Lighting assembly according to claim 1,
wherein the lighting device comprises a second light source configured to emit green-blue light.

6. Lighting assembly according to claim 1,
wherein the lighting device comprises at least a third light source configured to emit light mainly in the spectral range from green to red light.

7. Lighting assembly according to claim 1,
wherein the at least one third light source comprises a conversion element configured to mainly fully convert blue light to lower energy light.

8. Lighting assembly according to claim 1,
wherein the melanopic effect of the total light emitted by the lighting device is adjustable by changing the melanopic effect factor of the blue light, without changing the chromaticity coordinate of the blue light or the whiteness of the white light for this purpose.

9. Lighting assembly according to claim 1,
wherein the lighting device is intended to emit the blue light in the direction of a ceiling of a room.

10. Lighting assembly according to claim 1,
wherein the lighting device is arranged to emit the white light in a direction away from a ceiling of a room.
